(19) United States
(12) Patent Application Publication
(10) Pub. No.: US 2026/0153482 A1
(43) Pub. Date: Jun. 4, 2026

(54) An Airborne Detection Device and Operational Method for Cracks in Concrete Infrastructure Structures

(71) Applicant: Shandong University, Jinan (CN)

(72) Inventors: JiPeng WANG, Jinan (CN); HuanXiang QIU, Jinan (CN); Hu ZHAI, Jinan (CN); Nan LI, Jinan (CN); XiaoHui CHEN, Jinan (CN); Yong LIU, Jinan (CN); Lin WU, Tianjin (CN); RuoYin XU, Jinan (CN); Chao GAO, Jinan (CN); Song MA, Jinan (CN)

(73) Assignees: Shandong University, Jinan (CN); Shandong Bowee Vision Information Technology Co., Ltd, Jinan (CN)

(21) Appl. No.: 18/976,392

(22) Filed: Dec. 11, 2024

(30) Foreign Application Priority Data
Jun. 4, 2024 (CN) 202410715082.1

(51) Int. Cl. G01N 29/06; B64U 20/87; B64U 101/26; G01N 29/22; G01N 33/38; G06T 7/00

(52) U.S. Cl. CPC G01N 29/069; B64U 20/87; G01N 29/225; G06T 7/0004; B64U 2101/26; G01N 33/383; G06T 2207/20084; G06T 2207/30132

(57) ABSTRACT

An airborne detection device and an operational method for cracks in concrete infrastructure structures include a multi-rotor UAV equipped with an ultrasonic mechanism for collecting concrete crack depth information and an imaging system for marking crack locations, capturing crack images, and extracting crack morphology information from these images. By integrating crack depth information with crack morphology data, the system generates a three-dimensional structure of the crack. The airborne detection device and method are suitable for the rapid inspection of large-scale concrete infrastructure (such as bridges, dams, buildings, etc.) in outdoor environments. By combining image recognition technology with ultrasonic techniques, it enables three-dimensional visualization of concrete cracks, providing reliable support for subsequent crack treatment decision-making.

### CROSS REFERENCE TO THE RELATED APPLICATIONS

This application is based upon and claims priority to Chinese Patent Application No. 202410715082.1, filed on Jun. 4, 2024, the entire contents of which are incorporated herein by reference.

### TECHNICAL FIELD

The invention belongs to the technical field of concrete crack detection, in particular to An Airborne Detection Device and Operational Method for Cracks in Concrete Infrastructure Structures.

### BACKGROUND

Since the second decade of this century, the whole world has significantly increased investment in the field of infrastructure construction. To better optimize the layout of infrastructure across the whole world, there has been a strong push to develop a new intelligent infrastructure system. Consequently, a series of representative projects in hydraulic engineering, transportation engineering, and construction engineering have been launched and implemented. As many large-scale infrastructure projects have gradually come into operation, there has been widespread attention on the research and development of intelligent operation and maintenance techniques to ensure these infrastructures fully realize their social and economic benefits.

Concrete is the most widely used construction material in the fields of hydraulic engineering, transportation engineering, and construction engineering. Due to the inherent characteristics of the material, it is highly susceptible to cracking. The presence of structural cracks can severely impact the functionality, safety, and stability of infrastructure. Therefore, the ability to quickly, accurately identify, assess, and detect structural cracks is of paramount importance.

However, the current maintenance and operation of concrete infrastructure still predominantly rely on manual labor, which leads to long work-term and low efficiency. Additionally, workers often have to operate in elevated positions for extended periods while carrying various detection devices, making it challenging to ensure their safety during the process.

To address these issues, existing techniques have further developed the following unmanned aerial vehicle (UAV)-based inspection techniques:

CN201910502811.4 inventes a UAV-based concrete crack detection device and method, which includes the UAV body. This invention also incorporates an adsorption mechanism to attach the UAV body to the concrete surface where the crack is to be inspected, as well as a crack detection structure. The crack detection mechanism consists of a rotary drive motor fixed to the UAV body and a camera and measuring ruler mounted on the side of the UAV body facing the crack to be inspected. The output end of the rotary drive motor is connected to the measuring ruler via a rotating shaft, allowing the measuring ruler to conform closely to the concrete surface at the location of the crack.

It can be observed that the existing method, which relies on a camera mounted on a UAV to photograph the concrete surface, has the following drawbacks:
1. It only uses the camera to capture images of the crack location and subsequently obtains length and width data within the plane based on the captured images. This approach fails to acquire depth data, resulting in a limited range of data types from the detection instruments and reduced detection accuracy. Additionally, the method cannot assess the crack's depth, making it impossible to obtain the three-dimensional structure of the crack;
2. The process requires the UAV to adhere to the surface for positioning while taking photographs. Due to the lengthy time required for both suction and release, the method suffers from low efficiency. Additionally, since the system cannot adhere to sloped surfaces, it is limited to detecting concrete surfaces directly above the UAV's horizontal plane, making it unsuitable for inspecting concrete structures with various inclinations.

### SUMMARY

To address these issues, this invention provides an airborne detection device and operational method for cracks in concrete infrastructure structures. It is designed for real-time, rapid inspection of large-scale concrete structures (such as bridges, dams, and buildings) in outdoor environments. Upon detecting cracks, the system integrates ultrasonic data and image recognition technique to obtain the three-dimensional structure of the cracks, thereby providing reliable support for subsequent crack treatment and decision-making processes.

To achieve the aforementioned objectives, this invention provides an airborne detection device and operational method for cracks in concrete infrastructure structures. The system includes a multi-rotor UAV, which is equipped with an ultrasonic mechanism for collecting depth information of concrete cracks, and a camera mechanism for marking crack locations, capturing crack images, and extracting crack morphology information based on the captured images;

The camera mechanism and ultrasonic mechanism are both mounted at the bottom of the multi-rotor UAV via a mounting bracket. The ultrasonic mechanism includes a two-degree-of-freedom rotatable mechanical arm attached to the mounting bracket, an ultrasonic generator located at the end of the rotatable mechanical arm, and a transducer positioned on the front-end of the rotatable mechanical arm. The ultrasonic generator is electrically connected to the transducer and to the UAV. Additionally, the ultrasonic generator is in communication with the UAV, which, in turn, communicates with a remote control terminal.

Preferably, the rotatable mechanical arm includes the first arm and the second arm. One end of the first arm is fixedly connected to an accommodation box that houses the ultrasonic mechanism. The accommodation box is slidably mounted on a base platform and has L-shaped sliding plates at both ends of its bottom. The other end of the first arm is connected to the second arm via a center of gravity angle adjustment component. The end of the second arm, distal to the center of gravity angle adjustment component, is connected to the transducer via a rotation distance adjustment component;

The top of the base platform is provided with a T-shaped sliding groove. The bottom of the accommodation box is slidably connected to the T-shaped sliding groove via the L-shaped sliding plates. The bottom of the base platform is fixed to the UAV's landing gear via a limiting ring.

Preferably, the center of gravity angle adjustment component includes the first drive motor, the first drive gear, the first drive rack, and the U-shaped positioning seat fixed to the base platform via a suspension rod. The top of the U-shaped positioning seat is pivotally connected to the second arm, and the connection between the second arm and the U-shaped positioning seat is equipped with the first drive gear. The shaft of the first drive gear is connected to the output shaft of the first drive motor, which is fixed within the U-shaped positioning seat. The first drive gear meshes with the first drive rack, and the first drive rack is fixed to one end of the first arm, close to the second arm.

Preferably, the rotation distance adjustment component includes a rotating part and a distance adjustment part connected to the output end of the rotating part. The distance adjustment part is equipped with two transducers positioned relative to each other;

The rotating part includes the second drive motor fixed at the end of the second arm, opposite the center of gravity angle adjustment component. The second drive motor is used to adjust the line connecting the two transducers on the distance adjustment part so that it becomes perpendicular to the line connecting the two ends of the crack;

The distance adjustment part includes the third drive motor, the second drive gear connected to the output end of the third drive motor, and two second drive racks symmetrically engaged with the second drive gear on either side. Each of the two second drive racks has a transducer fixed to it. Symmetrically positioned on the outer sides of the two second drive racks are two grooves, each containing one end of an L-shaped sliding plate. The other end of each L-shaped sliding plate is connected to a sleeve. Inside the sleeve, a transducer is mounted and cushioned by a spring, ensuring stable positioning.

Preferably, the camera mechanism includes a gimbal, a camera fixed to the gimbal, and a laser rangefinder sensor attached to the camera. The center of the camera lens is equipped with crosshairs, which consist of a horizontal reference line and a vertical reference line that are perpendicular to each other;

The gimbal, camera, and laser rangefinder sensor are all in communication with the remote control terminal;

The processing platform module within the remote control terminal is equipped with a point calibration function and a concrete crack morphology recognition model for extracting crack shape information. The concrete crack morphology recognition model includes an optimized YOLO model, which has been enhanced using OpenCV image processing algorithms and Z-Score threshold detection algorithms. The YOLO model is built on the Pytorch deep learning framework and utilizes convolutional neural networks (CNNs);

The crack morphology information includes crack length, crack width, crack inclination angle, and crack contour.

An airborne detection device and operational method for cracks in concrete infrastructure structures includes the following steps:
S1. Surface Inspection: The remote control terminal operates the multi-rotor UAV, equipped with the camera mechanism and ultrasonic mechanism, to take off and approach the target concrete structure area. The gimbal is adjusted to align the camera's pitch angle so that the central axis of the camera lens is perpendicular to the surface of the concrete structure to be inspected. The UAV then performs a horizontal side flight parallel to the concrete surface, capturing images of the structure during flight;
S2. Extract surface information of the target concrete structure:
S21. The remote control terminal's processing platform, equipped with the concrete crack morphology recognition model, uses real-time video transmitted by the UAV's camera during side flight to detect the presence of cracks. If a crack is detected, the remote control terminal directs the UAV to hover and capture detailed images of the crack, which are then transmitted back to the processing platform. The platform uses its point calibration function to mark the exact location of the crack;
S22. Based on the gimbal's control of the camera's pitch angle, the remaining angle is calculated and used as the inclination angle of the surface of the target concrete structure;
S23. The remote control terminal's processing platform analyzes the captured crack images to identify and extract the angle between the line connecting the two ends of the concrete crack and the crosshairs. The angle between this line and the horizontal reference line is considered the crack inclination angle. If this inclination angle is less than a predetermined value, the remaining angle between the line connecting the crack ends and the vertical reference line is used as the crack inclination angle;
S3. Concrete Structure Crack Depth Detection:
S31. Direct the multi-rotor UAV to fly to the previously calibrated crack location. Adjust the two-degree-of-freedom rotatable mechanical arm based on the concrete surface inclination angle so that the two transducers are oriented perpendicular to the surface of the concrete structure. Additionally, adjust the alignment of the transducers to ensure that their connecting line is perpendicular to the line connecting the two ends of the crack, taking into account the crack inclination angle;
S32. Direct the multi-rotor UAV to approach the crack in the concrete structure. Using the concrete crack depth ultrasonic plane testing method, perform sequential measurements with multiple sets of different transducer spacings for both non-crack and across-crack detection. For each measurement, ensure that the two transducers are in close contact with the concrete surface. Emit ultrasonic signals into the concrete structure, and upon reflection from the crack's inner walls, the signals are returned to the remote control terminal's processing platform. The processing platform calculates the crack depth based on the ultrasonic plane testing method;
S4. The remote control terminal's processing platform processes the crack images to extract data on crack length, width, inclination angle, and contour. Using a three-dimensional visualization program developed in MATLAB, the platform integrates these data into a common spatial coordinate system. Based on the contour's spatial distribution, it performs smooth curve fitting on the datasets for crack width, length, and depth to determine the spatial distribution pattern of the crack. Finally, the platform generates a three-dimensional surface representation of the crack according to its contour;
S5. Output the completed three-dimensional visual model of the concrete crack and the identified crack morphology images.

Preferably, in step S22, during the adjustment of the camera's pitch angle by the gimbal, the laser rangefinder sensor is used to measure the distance between the camera and the surface of the concrete structure in real-time. The distance values are continuously compared, and when the distance value is minimized, it is determined that the central axis of the camera lens is perpendicular to the surface of the concrete structure.

Preferably, step S32 includes the following specific steps:
S321. Direct the multi-rotor UAV to fly to the surface of the concrete structure to be inspected. The first drive motor rotates the first drive gear, which adjusts the pitch angle of the second arm until the two transducers are oriented perpendicular to the concrete surface. Once the correct orientation is achieved, the first drive motor is turned off. During this process, the first drive rack moves the first arm and accommodation box horizontally along the T-shaped sliding groove on the base platform, ensuring dynamic stability of the center of gravity;
S322. Activate the second drive motor to adjust the angle of the line connecting the two transducers until it is perpendicular to the line connecting the two ends of the crack. Once the alignment is correct, turn off the second drive motor;
S323. Direct the multi-rotor UAV to approach the crack in the concrete structure. Activate the third drive motor to rotate the second drive gear, which causes the two transducers to move towards or away from each other under the action of the second drive rack. Adjust the transducer spacing to multiple different distances according to the ultrasonic plane testing method. Once the desired spacing is achieved, turn off the third drive motor;
S324. Direct the multi-rotor UAV to slowly approach and ensure that the two transducers are firmly in contact with the surface of the object to be inspected. The remote control terminal operates the ultrasonic generator to emit ultrasonic signals from the transducers into the concrete. The reflected ultrasonic signals are received and transmitted back. The processing platform at the remote control terminal processes the real-time ultrasonic detection signals based on the ultrasonic plane testing method to determine the crack depth at the calibrated location;
S325. Direct the multi-rotor UAV to move away from the concrete surface and fly to the next calibrated location. Perform non-crack and across-crack ultrasonic measurement according to the principles of the ultrasonic plane testing method until all calibrated points of the crack have been inspected.

Preferably, in step S324, the crack depth calculation formula is as follows:

$${h}_{ci} = \frac{{l}_{i}}{2} \sqrt{{( \frac{{t}_{i}^{0} v}{{l}_{i}} )}^{2} - 1}$$

$${m}_{hc} = \frac{1}{n} * {\sum }_{i = 1}^{n} {h}_{ci}$$

where:
h_{ci} is the crack depth value at the i^{th} point, in mm;
lᵢ is the actual ultrasonic propagation distance at the i^{th} point during non-crack measurement, in mm;

$${t}_{i}^{0}$$

is the acoustic time value for the i^{th} point during across-crack measurement, in μs;
v is the speed of sound in concrete during non-crack measurement, in km/s;
m_{hc} is the average crack depth calculated from all measurement points, in mm;
n is the number of measurement points.

This invention has the following beneficial effects:
1. The system synchronously combines ultrasonic detection with visual inspection to integrate crack depth, orientation, and width into a cohesive three-dimensional crack model. Compared to traditional device, this approach offers a more comprehensive and intuitive assessment of cracks, providing reliable support for subsequent crack treatment decisions;
2. By adjusting the distance between the two transducers using the distance adjustment part and performing multiple tests with interpolated distances to closely approach the phase reversal position (where phase reversal typically occurs during across-crack detection in ultrasonic plane testing method), the system ensures more accurate results. Taking the average of these measurements enhances precision, eliminates redundant steps in repetitive testing, and improves overall detection accuracy and operational efficiency;
3. The center of gravity is dynamically adjusted using the center of gravity angle adjustment component. As the second arm is lowered, the first arm and accommodation box extend horizontally forward. Conversely, as the second arm is raised, the first arm and accommodation box retract horizontally. This dynamic adjustment maintains overall balance and enhances safety;
4. By coordinating the operation of various drive motors, the system enables mode switching for the rotatable mechanical arm and detection components. This adaptability makes it suitable for inspecting a wide range of concrete infrastructure designs and structural defects, accommodating different design configurations and inspection needs
5. Most components of the system, other than the multi-rotor UAV platform, are made from high-strength, lightweight carbon fiber. This material choice reduces material usage and maintenance costs compared to traditional equipment, extends the service life of the device, and effectively controls the UAV's payload and operational duration while ensuring structural stability. Additionally, it enhances the device's durability under outdoor conditions such as sun exposure.

### BRIEF DESCRIPTION OF THE DRAWINGS

The following detailed description of the technical solution of the present invention will be provided with reference to the accompanying drawings and examples.

FIG. 1 is a schematic diagram illustrating the operation of the airborne detection device for cracks in concrete infrastructure structures of the present invention;

FIG. 2 is a side view of the horizontal working state of the airborne detection device for cracks in concrete infrastructure structures of the present invention;

FIG. 3 is a top view of the airborne detection device for cracks in concrete infrastructure structures of the present invention;

FIG. 4 is a schematic diagram illustrating the connection between the first arm and the second arm of the airborne detection device for cracks in concrete infrastructure structures of the present invention;

FIG. 5 is a cross-sectional view of the distance adjustment part of the airborne detection device for cracks in concrete infrastructure structures of the present invention;

FIG. 6 is a structural schematic diagram of the distance adjustment part of the airborne detection device for cracks in concrete infrastructure structures of the present invention;

FIG. 7 is a schematic diagram of the crosshair of the airborne detection device for cracks in concrete infrastructure structures of the present invention.

FIG. 8 is a schematic diagram illustrating the ultrasonic plane testing method used in the working method of the airborne detection device for cracks in concrete infrastructure structures of the present invention;

FIGS. 9A-9B are schematic diagrams of the detection results obtained using the method of the airborne detection device for cracks in concrete infrastructure structures of the present invention. FIG. 9A shows the crack morphology, and FIG. 9B displays the three-dimensional structural view of the crack.

Here is the labeled list for the figures: 1. Concrete Structure; 2. Multi-Rotor UAV; 21: Mounting Frame; 3. Camera System; 31: Camera; 32: Gimbal; 33: Laser Distance Sensor; 34: Crosshair; 4. Ultrasonic System; 41: Ultrasonic Generator; 42: Transducer; 5. Rotatable Mechanical Arm; 51: First Arm; 52: Second Arm; 6. Center of Gravity Adjustment Component; 61: First Drive Gear; 62: First Drive Rack; 7. Rotation Distance Adjustment Component; 71: Second Drive Motor; 72: Third Drive Motor; 73: Second Drive Rack; 74: Second Drive Gear; 75: Sleeve; 76: L-Shaped Slide Plate.

### DETAILED DESCRIPTION OF THE EMBODIMENTS

To provide a clearer understanding of the objectives, technical solutions, and advantages of the embodiments disclosed in the present invention, the following detailed description of the embodiments will be given with reference to the accompanying drawings. It should be understood that the specific embodiments described here are intended to illustrate the embodiments of the present invention and are not intended to limit the scope of the invention. All other embodiments that a person skilled in the art may derive from the embodiments in this application without making inventive efforts are within the scope of protection of this application. The examples of the embodiments are shown in the accompanying drawings, where the same or similar reference numbers throughout the figures represent the same or similar components or components with the same or similar functions.

It should be noted that the terms “comprise” and “include” and their variations are intended to cover non-exclusive inclusions. For example, a process, method, system, product, or server that includes a series of steps or units is not limited to the explicitly listed steps or units, but may also include other steps or units that are not explicitly listed or are inherent to these processes, methods, products, or devices.

Similar reference numbers and letters in the following drawings represent similar items. Therefore, once an item is defined in one drawing, it does not need to be further defined or explained in the subsequent drawings.

In the description of the present invention, it should be noted that terms such as “upper,” “lower,” “inner,” and “outer” refer to positional or directional relationships based on the orientation or positioning shown in the drawings, or the conventional orientation or positioning of the invention when in use. These terms are used for ease of description and simplification and do not indicate or imply that the device or component must have a specific orientation or be constructed and operated in a particular orientation. Therefore, they should not be interpreted as limiting the scope of the invention.

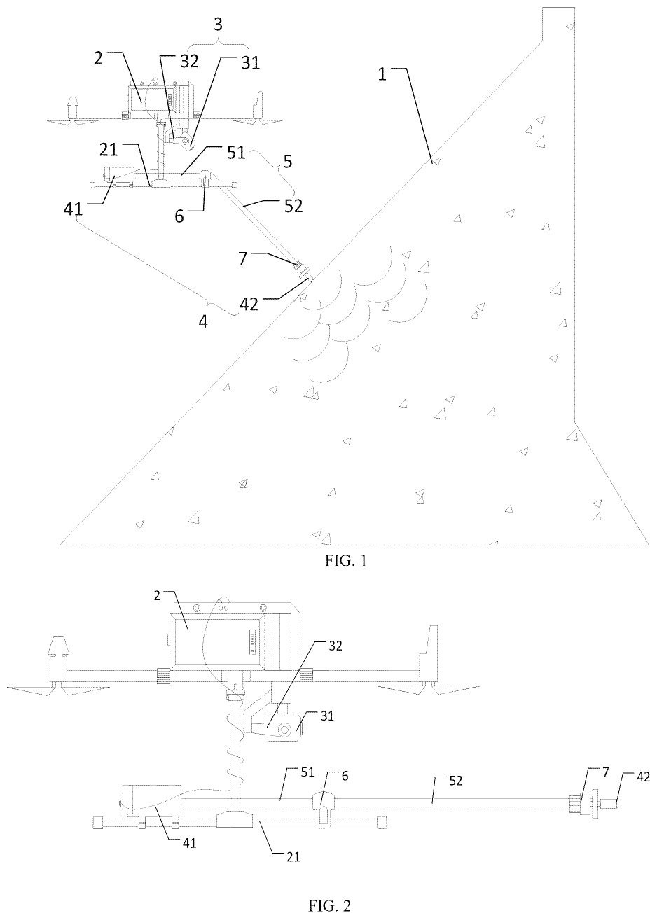

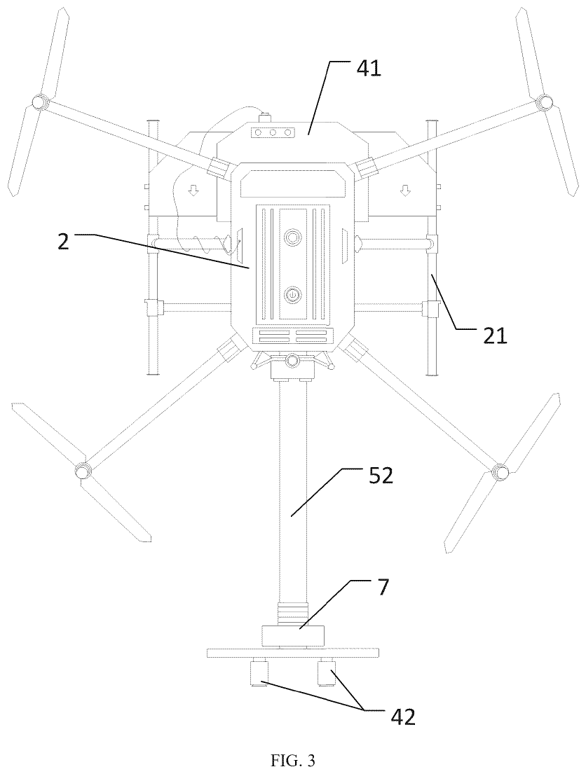

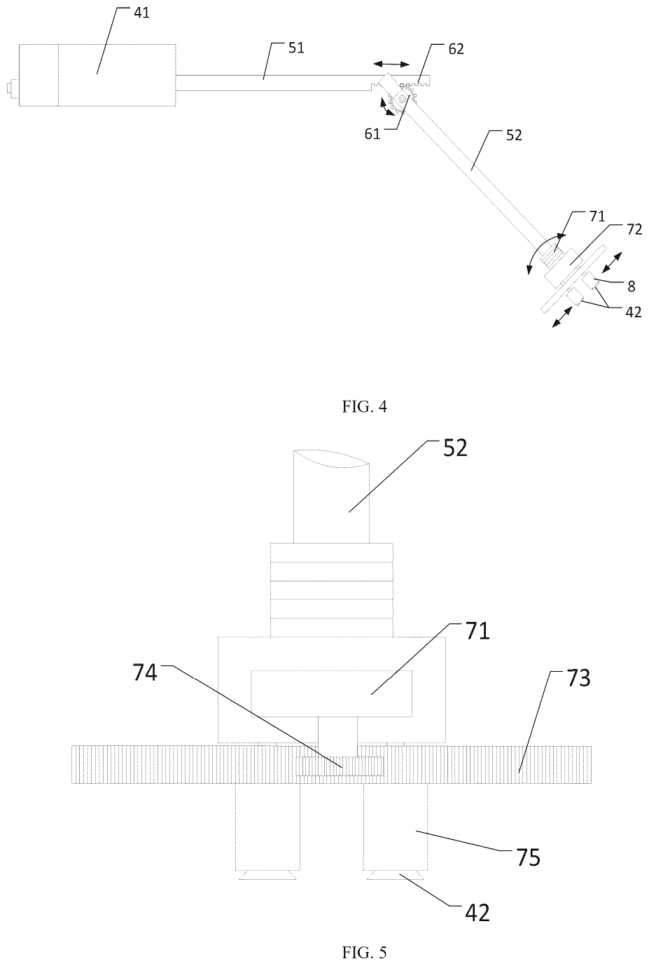

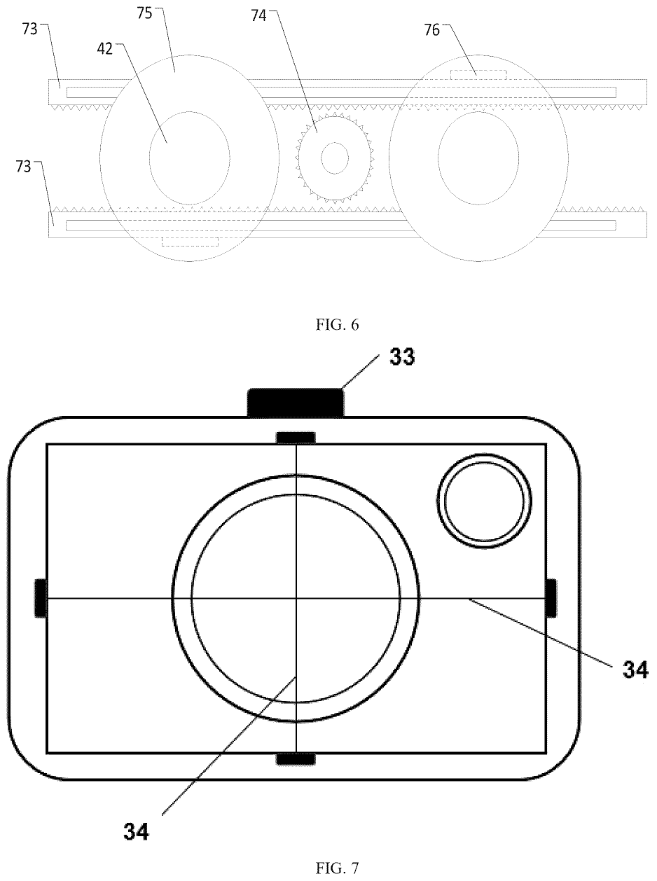

As shown in FIGS. 1-7, an airborne detection device for cracks in concrete infrastructure structures includes a multi-rotor UAV, which is equipped with an ultrasonic mechanism for collecting depth information of concrete cracks, and a camera mechanism for marking crack locations, capturing crack images, and extracting crack morphology information based on the captured images. The camera mechanism and ultrasonic mechanism are both mounted at the bottom of the multi-rotor UAV via a mounting bracket. The ultrasonic mechanism includes a two-degree-of-freedom rotatable mechanical arm attached to the mounting bracket, an ultrasonic generator located at the end of the rotatable mechanical arm, and a transducer positioned on the front-end of the rotatable mechanical arm. The ultrasonic generator is electrically connected to the transducer and to the UAV. Additionally, the ultrasonic generator is in communication with the UAV, which, in turn, communicates with a remote control terminal. This device facilitates intelligent maintenance of concrete infrastructure by enabling rapid, real-time detection of concrete cracks and the construction of a three-dimensional model of the cracks.

The rotatable mechanical arm includes the first arm and the second arm. One end of the first arm is fixedly connected to an accommodation box that houses the ultrasonic mechanism. The accommodation box is slidably mounted on a base platform and has L-shaped sliding plates at both ends of its bottom. The other end of the first arm is connected to the second arm via a center of gravity angle adjustment component. The end of the second arm, distal to the center of gravity angle adjustment component, is connected to the transducer via a rotation distance adjustment component. The top of the base platform is provided with a T-shaped sliding groove. The bottom of the accommodation box is slidably connected to the T-shaped sliding groove via the L-shaped sliding plates. The bottom of the base platform is fixed to the UAV's landing gear via a limiting ring.

The center of gravity angle adjustment component includes the first drive motor, the first drive gear, the first drive rack, and the U-shaped positioning seat fixed to the base platform via a suspension rod. The top of the U-shaped positioning seat is pivotally connected to the second arm, and the connection between the second arm and the U-shaped positioning seat is equipped with the first drive gear. The shaft of the first drive gear is connected to the output shaft of the first drive motor, which is fixed within the U-shaped positioning seat. The first drive gear meshes with the first drive rack, and the first drive rack is fixed to one end of the first arm, close to the second arm.

The rotation distance adjustment component includes a rotating part and a distance adjustment part connected to the output end of the rotating part. The distance adjustment part is equipped with two transducers positioned relative to each other. The rotating part includes the second drive motor fixed at the end of the second arm, opposite the center of gravity angle adjustment component. The second drive motor is used to adjust the line connecting the two transducers on the distance adjustment part so that it becomes perpendicular to the line connecting the two ends of the crack. The distance adjustment part includes the third drive motor, the second drive gear connected to the output end of the third drive motor, and two second drive racks symmetrically engaged with the second drive gear on either side. Each of the two second drive racks has a transducer fixed to it. The outer side of the transducer is fitted with a protective cover, and a cushioning spring is positioned between the transducer and the protective cover to buffer the impact when making contact with the concrete structure surface. Symmetrically positioned on the outer sides of the two second drive racks are two grooves, each containing one end of an L-shaped sliding plate. The other end of each L-shaped sliding plate is connected to a sleeve. Inside the sleeve, a transducer is mounted and cushioned by a spring, ensuring stable positioning. This enables the use of the third drive motor to rotate the second drive gear, which in turn drives the second drive rack to move. During this movement, the two transducers are either moved towards or away from each other, thereby adjusting the distance between the two transducers.

The camera mechanism includes a gimbal, a camera fixed to the gimbal, and a laser rangefinder sensor attached to the camera. The center of the camera lens is equipped with crosshairs, which consist of a horizontal reference line and a vertical reference line that are perpendicular to each other. The gimbal, camera, and laser rangefinder sensor are all in communication with the remote control terminal. The processing platform module within the remote control terminal is equipped with a point calibration function and a concrete crack morphology recognition model for extracting crack shape information. The concrete crack morphology recognition model includes an optimized YOLO model, which has been enhanced using OpenCV image processing algorithms and Z-Score threshold detection algorithms. The YOLO model is built on the Pytorch deep learning framework and utilizes convolutional neural networks (CNNs). The crack morphology information includes crack length, crack width, crack inclination angle, and crack contour.

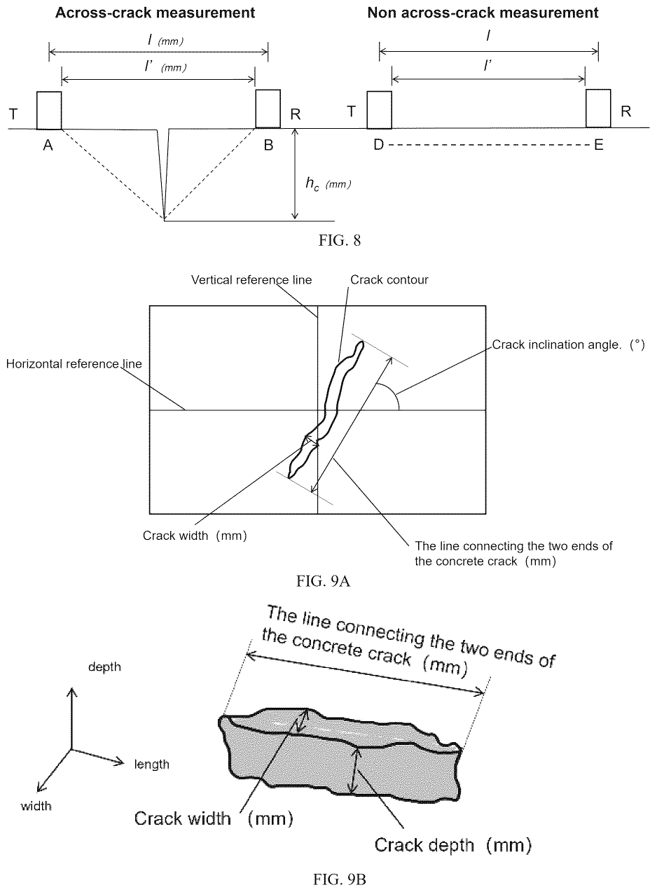

As shown in FIGS. 8-9B, a method for operating an airborne crack detection device for concrete infrastructure structures includes the following steps:
S1. Surface Inspection: The remote control terminal operates the multi-rotor UAV, equipped with the camera mechanism and ultrasonic mechanism, to take off and approach the target concrete structure area. The gimbal is adjusted to align the camera's pitch angle so that the central axis of the camera lens is perpendicular to the surface of the concrete structure to be inspected. The UAV then performs a horizontal side flight parallel to the concrete surface, capturing images of the structure during flight;
Before Step S1, the following step is included: Model Initialization:
Step 1: Data Collection: Collect images of concrete structure cracks to obtain a dataset. The dataset is then divided into training, validation, and test sets in a ratio of 3:2:1;
Step 2: Build the Concrete Crack Appearance Recognition Model: Develop a model for recognizing the appearance of concrete cracks;
Step 3: Model Training: Input the training dataset into the concrete crack appearance recognition model and train it until the recognition results achieve an error margin within the millimeter range compared to manual inspection results;
Step 4: Model Evaluation: Input the validation dataset into the trained concrete crack appearance recognition model to assess whether the evaluation metrics meet the required standards. If the metrics are satisfactory, proceed to Step 5; if not, return to Step 3;
Step 5: Embed the evaluated concrete crack appearance recognition model into the remote control platform;
After Step S1, the following steps are included:
Step 1: Data Cleaning. Sequentially perform denoising, missing value imputation, outlier handling, and dimensionality reduction on the captured crack image data to obtain the raw dataset;
Step 2: Data Annotation. Annotate the raw dataset to obtain feature sets;
Step 3: Feature Selection. Select the most useful subset of features from the feature set for the machine learning problem. In this embodiment, the most useful subset of features consists of images with clear crack contours and no surrounding attachments;
Step 4: Feature Extraction. Convert the feature subset into numerical features and input these numerical features into the concrete crack appearance morphology recognition model;
Step 5: Use the concrete crack appearance morphology recognition model to read the crack morphology information. Compare the retrieved crack morphology information with a predefined safety range. If the information exceeds the safety range, output the crack morphology information; otherwise, discard it;
S2. Extract surface information of the target concrete structure:
S21. The remote control terminal's processing platform, equipped with the concrete crack morphology recognition model, uses real-time video transmitted by the UAV's camera during side flight to detect the presence of cracks. If a crack is detected, the remote control terminal directs the UAV to hover and capture detailed images of the crack, which are then transmitted back to the processing platform. The platform uses its point calibration function to mark the exact location of the crack;
S22. Based on the gimbal's control of the camera's pitch angle, the remaining angle is calculated and used as the inclination angle of the surface of the target concrete structure;
In step S22, during the adjustment of the camera's pitch angle by the gimbal, a laser distance sensor is used to measure the distance between the camera and the surface of the concrete structure to be inspected in real-time. The distance values are continuously compared, and when the distance value is minimized, it is determined that the optical axis of the camera lens is perpendicular to the surface of the concrete structure being inspected.
S23. The remote control terminal's processing platform analyzes the captured crack images to identify and extract the angle between the line connecting the two ends of the concrete crack and the crosshairs. The angle between this line and the horizontal reference line is considered the crack inclination angle. If this inclination angle is less than a predetermined value, the remaining angle between the line connecting the crack ends and the vertical reference line is used as the crack inclination angle
The crack length and width from the same set of crack images are used as input variables, with the maximum length and width within the image serving as output targets. The line with the maximum length connecting the ends of the crack is designated as the crack's tilt line. The angle between this tilt line and the horizontal reference line of the crosshair, or the complementary angle with the vertical reference line, is used to determine the crack's tilt angle.
S3: Concrete Structure Crack Depth Detection:
S31. Direct the multi-rotor UAV to fly to the previously calibrated crack location. Adjust the two-degree-of-freedom rotatable mechanical arm based on the concrete surface inclination angle so that the two transducers are oriented perpendicular to the surface of the concrete structure. Additionally, adjust the alignment of the transducers to ensure that their connecting line is perpendicular to the line connecting the two ends of the crack, taking into account the crack inclination angle;
S32. Direct the multi-rotor UAV to approach the crack in the concrete structure. Using the concrete crack depth ultrasonic plane testing method, perform sequential measurements with multiple sets of different transducer spacings for both non-crack and across-crack detection. For each measurement, ensure that the two transducers are in close contact with the concrete surface. Emit ultrasonic signals into the concrete structure, and upon reflection from the crack's inner walls, the signals are returned to the remote control terminal's processing platform. The processing platform calculates the crack depth based on the ultrasonic plane testing method. Step S32 includes the following specific steps:
S321. Direct the multi-rotor UAV to fly to the surface of the concrete structure to be inspected. The first drive motor rotates the first drive gear, which adjusts the pitch angle of the second arm until the two transducers are oriented perpendicular to the concrete surface. Once the correct orientation is achieved, the first drive motor is turned off. During this process, the first drive rack moves the first arm and accommodation box horizontally along the T-shaped sliding groove on the base platform, ensuring dynamic stability of the center of gravity;
S322. Activate the second drive motor to adjust the angle of the line connecting the two transducers until it is perpendicular to the line connecting the ends of the crack. Once the alignment is achieved, turn off the second drive motor;
S323. Direct the multi-rotor UAV to approach the crack in the concrete structure. Activate the third drive motor to rotate the second drive gear, which causes the two transducers to move towards or away from each other under the action of the second drive rack. Adjust the transducer spacing to multiple different distances according to the ultrasonic plane testing method. Once the desired spacing is achieved, turn off the third drive motor;
In this embodiment, the distance between the two transducers is set to a total of five different configurations. These include three distances based on the ultrasonic plane testing method: 50 mm, 100 mm, and 150 mm. Additionally, two interpolated distances of 75 mm and 125 mm are included to approximate the conditions near the occurrence of ultrasonic wave phase inversion, thereby enhancing the accuracy of the detection;
S324. Direct the multi-rotor UAV to slowly approach and ensure that the two transducers are firmly in contact with the surface of the object to be inspected. The remote control terminal operates the ultrasonic generator to emit ultrasonic signals from the transducers into the concrete. The reflected ultrasonic signals are received and transmitted back. The processing platform at the remote control terminal processes the real-time ultrasonic detection signals based on the ultrasonic plane testing method to determine the crack depth at the calibrated location.

In step S324, the formula for calculating crack depth is as follows:

$${h}_{ci} = \frac{{l}_{i}}{2} \sqrt{{( \frac{{t}_{i}^{0} v}{{l}_{i}} )}^{2} - 1}$$

$${m}_{hc} = \frac{1}{n} * {\sum }_{i = 1}^{n} {h}_{ci}$$

where:
h_{ci} is the crack depth value at the i^{th} point, in mm;
lᵢ is the actual ultrasonic propagation distance at the i^{th} point during non-crack measurement, in mm;

$${t}_{i}^{0}$$

is the acoustic time value for the i^{th} point during across-crack measurement, in μs;
v is the speed of sound in concrete during non-crack measurement, in km/s;
m_{hc} is the average crack depth calculated from all measurement points, in mm;
n is the number of measurement points.
S325. Direct the multi-rotor UAV to move away from the concrete surface and fly to the next calibrated location. Perform non-crack and across-crack ultrasonic measurement according to the principles of the ultrasonic plane testing method until all calibrated points of the crack have been inspected.
S4. The remote control terminal's processing platform processes the crack images to extract data on crack length, width, inclination angle, and contour. Using a three-dimensional visualization program developed in MATLAB, the platform integrates these data into a common spatial coordinate system. Based on the contour's spatial distribution, it performs smooth curve fitting on the datasets for crack width, length, and depth to determine the spatial distribution pattern of the crack. Finally, the platform generates a three-dimensional surface representation of the crack according to its contour;
S5. Output the completed three-dimensional visual model of the concrete cracks and the identified crack appearance images.

Thus, this invention utilizes the aforementioned airborne detection device and operational method for cracks in concrete infrastructure structures, which is suitable for rapid inspection of large-scale concrete infrastructures (such as bridges, dams, buildings, etc.) in outdoor environments. By integrating ultrasonic testing technology with image recognition technology, the invention obtains a three-dimensional structure of the cracks after detection, providing reliable support for subsequent crack treatment decisions.

Finally, it should be noted that the above embodiments are provided to illustrate the technical solutions of the present invention and are not intended to limit the invention. Although the invention has been described in detail with reference to the preferred embodiments, those skilled in the art will understand that modifications or equivalent substitutions can be made to the technical solutions of the invention. Such modifications or substitutions should not deviate from the spirit and scope of the technical solutions of the present invention.

What is claimed is:

1. An airborne detection device for cracks in concrete infrastructure structures, comprising a multi-rotor unmanned aerial vehicle (UAV), wherein the multi-rotor UAV is equipped with an ultrasonic mechanism for collecting depth information of concrete cracks, and a camera mechanism for marking crack locations, capturing crack images, and extracting crack morphology information based on captured crack images, and by integrating the depth information of the concrete cracks with the crack morphology information, the airborne detection device generates a three-dimensional structure of the cracks;
the camera mechanism and the ultrasonic mechanism are both mounted at a bottom of the multi-rotor UAV via a mounting bracket, the ultrasonic mechanism comprises a two-degree-of-freedom rotatable mechanical arm attached to the mounting bracket, an ultrasonic generator located at an end of the two-degree-of-freedom rotatable mechanical arm, and a transducer positioned at a base of the two-degree-of-freedom rotatable mechanical arm, the ultrasonic generator is electrically connected to the transducer and to the multi-rotor UAV, additionally, the ultrasonic generator is in communication with the multi-rotor UAV, and in turn, the multi-rotor UAV communicates with a remote control terminal;
the two-degree-of-freedom rotatable mechanical arm comprises a first arm and a second arm, a first end of the first arm is fixedly connected to an accommodation box, wherein the accommodation box houses the ultrasonic mechanism, the accommodation box is slidably mounted on a base platform and has L-shaped sliding plates at both ends of a bottom of the accommodation box, a second end of the first arm is connected to the second arm via a center of gravity angle adjustment component, and an end of the second arm, distal to the center of gravity angle adjustment component, is connected to the transducer via a rotation distance adjustment component; and
a top of the base platform is provided with a T-shaped sliding groove, the bottom of the accommodation box is slidably connected to the T-shaped sliding groove via the L-shaped sliding plates, and a bottom of the base platform is fixed to a landing gear of the multi-rotor UAV via a limiting ring.

2. The airborne detection device according to claim 1, wherein the center of gravity angle adjustment component comprises a first drive motor, a first drive gear, a first drive rack, and a U-shaped positioning seat fixed to the base platform via a suspension rod, a top of the U-shaped positioning seat is pivotally connected to the second arm, a connection between the second arm and the U-shaped positioning seat is equipped with the first drive gear, a shaft of the first drive gear is connected to an output shaft of the first drive motor, wherein the first drive motor is fixed within the U-shaped positioning seat, the first drive gear meshes with the first drive rack, and the first drive rack is fixed to the second end of the first arm, adjacent to the second arm.

3. The airborne detection device according to claim 2, wherein the rotation distance adjustment component comprises a rotating part and a distance adjustment part connected to an output end of the rotating part, and the distance adjustment part is equipped with two transducers positioned opposite to each other;
the rotating part comprises a second drive motor, wherein the second drive motor is fixed at the end of the second arm, opposite to the center of gravity angle adjustment component, the second drive motor is configured to adjust an alignment of the two transducers on the distance adjustment part, wherein a line connecting the two transducers is perpendicular to a line connecting two ends of the cracks; and
the distance adjustment part comprises a third drive motor, a second drive gear connected to an output end of the third drive motor, and two second drive racks, wherein the two second drive racks are symmetrically engaged with the second drive gear on both sides, two grooves are symmetrically positioned on outer sides of the two second drive racks, each of the two grooves houses a first end of each of the L-shaped sliding plates, a second end of each of the L-shaped sliding plates is connected to a sleeve, and the transducer is mounted within the sleeve and cushioned by a spring.

4. The airborne detection device according to claim 3, wherein the camera mechanism comprises a gimbal, a camera fixed to the gimbal, and a laser rangefinder sensor attached to the camera, a center of camera lens is equipped with crosshairs, wherein the crosshairs comprise a horizontal reference line and a vertical reference line, wherein the horizontal reference line and the vertical reference line are perpendicular to each other;
the gimbal, the camera, and the laser rangefinder sensor are all in communication with the remote control terminal;
a processing platform module within the remote control terminal is equipped with a point calibration function and a concrete crack morphology recognition model for extracting the crack morphology information, the concrete crack morphology recognition model comprises an optimized YOLO model, wherein the optimized YOLO model has been enhanced using OpenCV image processing algorithms and Z-Score threshold detection algorithms, and the optimized YOLO model is built on a Pytorch deep learning framework and utilizes convolutional neural networks (CNNs); and
the crack morphology information comprises crack length, crack width, crack inclination angle, and crack profile.

5. An operational method for the airborne detection device according to claim 4, comprising the following steps:
S1. surface inspecting: operating, by the remote control terminal, the multi-rotor UAV to take off and approach a target concrete structure area, adjusting the gimbal to align a pitch angle of the camera, wherein the multi-rotor UAV is equipped with the camera mechanism and the ultrasonic mechanism, and a central axis of the camera lens is perpendicular to a surface of a concrete structure to be inspected, performing a horizontal side flight parallel to the surface of the concrete structure to be inspected by the multi-rotor UAV, and capturing images of the concrete structure to be inspected during the horizontal side flight;
S2. extracting surface information of the concrete structure to be inspected:
S21. using, by the processing platform module of the remote control terminal, a real-time video transmitted by the camera of the multi-rotor UAV during the horizontal side flight to detect a presence of the cracks, wherein the processing platform module is equipped with the concrete crack morphology recognition model; when a crack is detected, directing, by the remote control terminal, the multi-rotor UAV to hover and capture detailed images of the crack, wherein the detailed images are transmitted back to the processing platform module, wherein the processing platform module applies the point calibration function to mark an exact location of the crack;
S22. based on the pitch angle of the camera of control of the gimbal, calculating a remaining angle and using the remaining angle as the inclination angle of the surface of the concrete structure to be inspected; and
S23. analyzing, by the processing platform module of the remote control terminal, the captured crack images to identify and extract an angle between the line connecting the two ends of the concrete cracks and the crosshairs, considering the angle between the line connecting the two ends of the cracks and the horizontal reference line as the crack inclination angle, and when the crack inclination angle is less than a predetermined value, considering a remaining angle between the line connecting the two ends of the cracks and the vertical reference line as the crack inclination angle;
S3. detecting concrete structure crack depth:
S31. directing the multi-rotor UAV to fly to a previously calibrated crack location, adjusting the two-degree-of-freedom rotatable mechanical arm based on the inclination angle of the surface of the concrete structure to be inspected, wherein the two transducers are oriented perpendicular to the surface of the concrete structure to be inspected, and additionally, adjusting the alignment of the two transducers to ensure that the line connecting the two transducers is perpendicular to the line connecting the two ends of the cracks, taking into account the crack inclination angle; and
S32. directing the multi-rotor UAV to approach the crack in the concrete structure to be inspected, using a concrete crack depth ultrasonic plane testing method, performing sequential measurements with a plurality of sets of different transducer spacings for both non-crack and across-crack detection, for each of the sequential measurements, ensuring that the two transducers are in close contact with the surface of the concrete structure to be inspected, emitting ultrasonic signals into the concrete structure to be inspected, upon a reflection from inner walls of the crack, returning the ultrasonic signals to the processing platform module of the remote control terminal, and calculating, by the processing platform module, the concrete structure crack depth based on the concrete crack depth ultrasonic plane testing method;
S4. processing, by the processing platform module of the remote control terminal, the captured crack images to extract data on the crack length, the crack width, the crack inclination angle and the crack profile, using a three-dimensional visualization program developed in MATLAB, integrating, by the processing platform module, the data on the crack length, the crack width, the crack inclination angle and the crack profile into a common spatial coordinate system, based on a spatial distribution of the crack profile, performing a smooth curve fitting on datasets for the crack width, the crack length, and the concrete structure crack depth to determine a spatial distribution pattern of the crack, and finally generating, by the processing platform module, a three-dimensional surface representation of the crack according to the crack profile; and
S5. outputting a completed three-dimensional visual model of the concrete cracks and identified crack appearance images.

6. The operational method according to claim 5, wherein in the step S22, during an adjustment of the pitch angle of the camera by the gimbal, the laser rangefinder sensor is configured to measure a distance between the camera and the surface of the concrete structure to be inspected in real-time, distance values are continuously compared, and when the distance value is minimized, it is determined that the central axis of the camera lens is perpendicular to the surface of the concrete structure to be inspected.

7. The operational method according to claim 5, wherein the step S32 comprises the following steps:
S321. directing the multi-rotor UAV to fly to the surface of the concrete structure to be inspected, and allowing the first drive motor to rotate the first drive gear, wherein the first drive gear adjusts a pitch angle of the second arm until the two transducers are oriented perpendicular to the surface of the concrete structure to be inspected, once a correct orientation is achieved, turning off the first drive motor, during a process of achieving the correct orientation, moving, by the first drive rack, the first arm and the accommodation box horizontally along the T-shaped sliding groove on the base platform, ensuring a dynamic stability of a center of gravity;
S322. activating the second drive motor to adjust an angle of the line connecting the two transducers until the line connecting the two transducers is perpendicular to the line connecting the two ends of the cracks, and once the alignment is correct, turning off the second drive motor;
S323. directing the multi-rotor UAV to approach the crack in the concrete structure, and activating the third drive motor to rotate the second drive gear, wherein the second drive gear causes the two transducers to move towards or away from each other under an action of the second drive rack, adjusting a transducer spacing to a plurality of different distances according to the concrete crack depth ultrasonic plane testing method, and once a desired spacing is achieved, turning off the third drive motor;
S324. directing the multi-rotor UAV to slowly approach and ensuring that the two transducers are firmly in contact with the surface of the concrete structure to be inspected, operating, by the remote control terminal, the ultrasonic generator to emit the ultrasonic signals from the two transducers into the concrete structure to be inspected, receiving reflected ultrasonic signals and transmitting the reflected ultrasonic signals back, and processing, by the processing platform module at the remote control terminal, real-time ultrasonic detection signals based on the concrete crack depth ultrasonic plane testing method to determine the concrete structure crack depth at the previously calibrated crack location; and
S325. directing the multi-rotor UAV to move away from the surface of the concrete structure to be inspected and fly to a next calibrated location, and performing non-crack and across-crack ultrasonic measurement according to principles of the concrete crack depth ultrasonic plane testing method until all calibrated points of the crack have been inspected.

8. The operational method according to claim 7, wherein in the step S324, a crack depth calculation formula is as follows:
$${h}_{ci} = \frac{{l}_{i}}{2} \sqrt{{( \frac{{t}_{i}^{0} v}{{l}_{i}} )}^{2} - 1} ;$$
$${m}_{hc} = \frac{1}{n} * {\sum }_{i = 1}^{n} {h}_{ci} ;$$
wherein:
h_{ci} is a crack depth value at an i^{th} point, in mm;
lᵢ is an actual ultrasonic propagation distance at the i^{th} point during the non-crack ultrasonic measurement, in mm;
$${t}_{i}^{0}$$
is an acoustic time value for the i^{th} point during the across-crack ultrasonic measurement, in μs;
v is a speed of sound in the concrete structure during the non-crack ultrasonic measurement, in km/s;
m_{hc} is an average crack depth calculated from all measurement points, in mm; and
n is a number of the measurement points.
